(19) United States
(12) Patent Application Publication
(10) Pub. No.: US 2005/0004427 A1
(43) Pub. Date: Jan. 6, 2005

(54) Prosthesis for use in the surgical treatment of urogenital prolapse and female urinary incontinence

(76) Inventor: Mauro Cervigni, Rome (IT)

Correspondence Address: AMS RESEARCH CORPORATION, 10700 BREN ROAD WEST, MINNETONKA, MN, 55343, US

(21) Appl. No.: 10/836,834

(22) Filed: Apr. 30, 2004

(30) Foreign Application Priority Data
Apr. 30, 2003 (IT) RM2003A000210

(51) Int. Cl. A61F 2/02

(52) U.S. Cl. 600/37

(57) ABSTRACT

A surgical prosthesis is disclosed.

### CROSS-REFERENCE TO RELATED APPLICATIONS

The present application claims the benefit of Italian Patent Application No. RM2003A000210 filed Apr. 30,2003, entitled, “Protesi da impiegarsi nella terapia chirurgica del prolasso urogenitale e dell'incontinenza urinaria femminile”, inventor Mauro Cervigni (the entire contents incorporated by reference herein).

### BACKGROUND

The invention concerns a prosthesis 1 which consists of a mesh made of non-absorbable or partially absorbable or biologic material. The central body 2 has a first portion 3 designed to be placed under the bladder neck B and the middle urethra C, and a second portion 4 to be applied to the body D and the base E of the bladder beneath the inferior part of the bladder; the portions are nominated first and second as this is the sequence of the direction of insertion of the prosthesis; a first pair of arms 5 and 6 which extend from each side of the first portion 3 of the central body 2, the said arms 5 and 6 designed to be placed in the obturator foramen F bilaterally and a second pair of arms 7 and 8 which extend from each side of the second section 4 of the central body 2, the said arms 7 and 8 designed to be applied to the lateral perineum to the labia major and laterally to the bulbocavernosus muscles.

### SUMMARY OF THE INVENTION

The present invention concerns a prosthesis for use in the surgical treatment of urogenital prolapse and female urinary incontinence.

More particularly the invention refers to a prosthesis to be used concomitantly in the surgical treatment of urogenital prolapse and female urinary incontinence allowing both problems to be resolved with a single operation.

The term urogenital prolapse refers to, in general, with reference to the physiology of the female pelvis, the inferior displacement of the uterus, the bladder and the rectum with consequent dislocation of the vaginal walls.

During the course of biological evolution, the humans assumed an erect posture Which has determined, from the anatomical point of view, reversal of the position of the viscera inside the so-called pelvic cavity.

In particular, the female pelvis must support the weight of the intraabdominal organs, without adequate counter-support from the pelvic structures.

In this context, the above-mentioned urogenital prolapse can be caused by many factors, amongst which any phenomenon which increases the intraabdominal pressure on the female pelvis, for example, traumatic sporting activity, long periods of time standing up, chronic cough, and obesity.

In addition, prolapse is more common in women who have had multiple deliveries but also where there is weakness and loss of tone in the structures which support the uterus, even in nulliparous women.

The consequences of a prolapse of lesser or greater severity lead to a general deterioration in the quality of life and provoke local circulatory disturbances, swelling of the external genitalia, back pain, feeling of weight in the lower abdomen and in serious cases ulceration and exudation and difficulty with micturition and defecation.

Moreover, an important consequence of prolapse is stress urinary incontinence, due to the descent of the anterior wall of the vagina, the urethra and the bladder neck.

Various types of urogenital prolapse may be distinguished, depending on the area involved, its precise position in this area and the stage of descent, that is: Stage I when the lower limit of the uterine cervix or the anterior vaginal wall reaches the middle of the vaginal canal without reaching the vulval orifice; Stage II when the uterine cervix or the anterior vaginal wall reaches the vulval orifice; Stage III when the lower extremity of the uterine cervix or the anterior vaginal wall protrudes from the vulva.

The term hysterocele refers to prolapse of the uterus; urethrocele when the urethra is prolapsed; cystocele when the bladder descends; rectocele when the rectum descents. In cases where more than one condition is present the terms are combined, for example urethro-cystocele or recto-cystocele.

A cystocele can occur because of detachment of the lateral or transverse endopelvic fascia or both (centro-lateral).

These different modalities are treated with specific surgical approaches which at times differ amongst themselves.

Operations for repair of lateral detachment of the fascia have been proposed, both abdominal (paravaginal repair) and vaginal (vaginal paravaginal repair); four points of the anterior vaginal wall may be surgically attached with four pairs of non-absorbable sutures to the retropubic area, in the so-called “four corner” technique. In central detachments, the approach consists of medializing the adjacent vaginal structures, in other words, the levator muscles and fascia, to raise the vagina, the bladder and sometimes the uterus.

With regards to the rectocele, the surgical approach includes the so-called posterior colporrhaphy, in other words the anchorage of the involved tissues to the medial part of the levator ani (Levator Myorrhaphy), or transanal treatment.

In spite of this variety of surgical therapies which allow the urogenital prolapse to be treated even at an early stage, when the various modalities are compared, the results are not satisfactory as there is a high rate of recurrence, up to 30%, after a follow-up period of 6 months to one year after the operation.

A recent analysis of data from the international literature has demonstrated that there is a recurrence rate of 20 to 78% for surgical repair of anterior vaginal wall prolapse with a follow-up of 6-48 months.

It is just as frequent that a patient treated for a prolapse in one area develops over time a prolapse in the non-treated area.

The cause of recurrence may be principally explained by the fact that any operation for repair demands, in the period of scar formation, a stabilization and reinforcement of the relevant connective structures. In reality, in many of these cases, the newly formed collagen constitutes a predisposition to successive descent of the surgically treated structures.

In fact, the connective tissue is weakened especially because in the typical age in Which prolapse occurs (per- or post-menopausal women) the fall in estrogens favors the formation of a weaker type of collagenous tissue.

From 1995 the Author of the present invention has introduced the use of synthetic mesh in the repair of genital prolapse, taking inspiration from the procedure proposed and carried out in General Surgery, that is to say the use of synthetic mesh in the repair of inguinal hernias. The recurrence rate of inguinal hernias has consequently fallen from 20-30% to 1-2%.

Equally the use of the synthetic mesh for repair of genital prolapse has drastically reduced the recurrence rate from 30-50% in the past to the present 3-6%.

The materials used are varied and the “ideal” material has not jet been identified, but Prolene® (interwoven synthetic monofilament: Polypropylene) comes closest to the ideal requisites of a biocompatible material. These materials, in order to reduce complications and long term sequelae to a minimum, must be applied “tension free”, in the sense that they must not cause traction or tension on the vaginal wall or bladder or rectum with consequent inevitable alteration of the functioning of these organs.

Often urogenital prolapse is also associated with urinary incontinence.

Stress urinary incontinence (SUI) is a condition that predominately affects women and is characterized by uncontrolled loss of urine during physical activity, or stress such as lifting a heavy object. The causes are various, amongst which: labor and delivery which determine trauma during the passage of the fetus in the vaginal canal; menopause; repeated pelvic surgery, for example hysterectomy. From a pathophysiological point of view the female urethral sphincter which is already weak is further weakened by stretching of the ligaments of support and relaxation of the perineal muscles (anal levator muscles).

In the past the various techniques for correcting SUI used the patient's own tissue to reinforce the sphinteric activity. A careful analysis of the literature demonstrates, however, that the recurrence rate can reach 15-20%. Thanks to the work of the Swedish School (Ulmsten) a new mini-invasive surgical procedure using Prolene® mesh has been introduced, applied with a “tension-free” technique under the middle-third of the urethra. The two ends of this tape are then transferred to the suprapubic area by 2 curved needles. A more recent variant uses the obturator foramen to stabilize the mesh under the urethra. In this way the support of the urethral sphincter is facilitated as “physiologically” as possible.

At the present time therefore a prosthesis is available for the treatment of SUI and a prosthesis is available for the treatment of genital prolapse but separate operations are required to resolve the above problems with consequent inconvenience for the patient. It is thus evident that there is a need for a new prosthesis which could resolve concurrently the problem of genital prolapse and SUI in a single operation.

The author of the present invention has prepared a prosthesis consisting of a mesh made of non-absorbable or partially absorbable material to apply using a “tension-free” technique, i.e., without forces of traction on the surrounding tissues, able to resolve both the above mentioned problems.

Thus this invention is a prosthesis to be used in the surgical treatment of urogenital prolapse and female urinary incontinence, constituting a mesh made of non-absorbable or partially absorbable or biological material. The central body has a first portion designed to be placed adjacent to bladder under the bladder neck and middle urethra; a second portion is to be placed in relation to the body and base of the bladder, under the inferior part of the bladder. The portions are described as first and second as this is the sequence of the direction of insertion of the prosthesis; a first pair of arms which extend from each side of the first portion of the central body, the said arms designed to be placed in the obturator foramen bilaterally and a second pair of arms which extend from each side of the second section of the central body, the said arms designed to be placed on the perineum, laterally to the labia major and passing through the bulbocavernosus muscles.

Preferably, the above named arms designated as first and second pair, are intended, at the respected ends not joined to the central body, to be a means for the attachment of single-use curved needles, that is the so-called means of attachment can be non-reusable in nature.

In particular, the said arms of the first pair of arms are angled with respect to the central body of the prosthesis in the opposite direction to the said direction of insertion of the actual prosthesis.

Continuing the description of the invention, the said arms of the second pair of arms are angled with respect to the central body of the prosthesis in the aforesaid direction of insertion of the prosthesis. Preferably, both the first and second pair of arms are coated with a nylon sheath to assist sliding over the tissues.

The prosthesis according to the present invention is made of a synthetic non-absorbable or partially absorbable. The present invention concerns a prosthesis for use in the surgical treatment of urogenital prolapse and female urinary incontinence.

The mesh, sterile and inert, which has bidirectional elastic extensibility and a resistance to traction greater than 10 kg/cm 2, being from 0.5 mm to 0.9 mm thick, preferably about 0.7 mm.

Regarding the intersections between the threads of the mesh, these are interconnected so that the mesh can be cut in any direction without unraveling.

The transverse dimensions of the pores, approximately rhomboid shaped, are between 1 mm and 3 mm.

Each thread is a polypropylene (C₃H₆) non-absorbable monofilament, more specifically an isotactic stereoisomer of polypropylene.

A type of mesh, which is particularly suitable for this type of application, is a mesh made of Prolene® monofilament, made by Ethicon® Inc. and marketed under the commercial name of polypropylene PROLENE® mesh.

As an alternative to the non-absorbable mesh described above, it is possible to use with the same efficacy a partially absorbable mesh or a biological material such as dermis, pericardium, or intestinal submucosa. Preferably, the mesh should be made of biological tissue in the central part and synthetic material in both the first and second pair of arms.

The surgical technique is now described below.

A longitudinal incision is made from 1 cm below the external urethral meatus to the anterior vaginal fornix. The underlying pubocervical fascia is then detached, arriving laterally to the ascending branch of the ischiopubic and laterally to the lateral fornix. Two (2) incisions are then carried out 1 cm above the clitoris in the inguinal folds and a curved needle is passed through the obturator foramen and exits at the sides of the bladder neck bilaterally. The apex of an arm of the prosthesis is then threaded into the needle (or attached using a Luer-Lock) and then pulled through the previous track. Two (2) further incisions of 1-2 cm are then executed laterally to the labia major at the level of the vaginal vestibule and with the same curved needle the bulbo-cavernosous muscle is penetrated and the end of the needle is brought out 2 cm posterior to the lateral fornix. A free flap of the mesh is placed under the base of the bladder and the vaginal opening is closed with a continuous suture. With a combined maneuver, applying traction to the arms which have been placed outside and on the anterior vaginal wall, the mesh is positioned as gently as possible, the excess tissue is then excised and the four (4) cutaneous openings are closed.

New findings regarding the etiology of pelvic descent have meant that the cystocele is considered to be a true “bladder hernia”. The alteration of and relaxation of the endopelvic fascia (“The anatomy of the pelvic floor”, J. O. DeLancey, Curr. Opin. Obstet. Gynecol. 1994 August; 6(4): 313-6) and the sagging of the muscles and ligaments (“Anatomy and biomechanics of genital prolapse”, J. O. DeLancey, Clin. Obstet. Gynecol. 1993 December; 36(4): 897-909) are the cause of the initial process of “bladder herniation”. The therapeutic strategy thus tends to be analogous to that of the hernia. The positioning of a “tension free” mesh tends to restore the altered support of the endopelvic fascia. The concept of the “tension free” technique has the further advantage of respecting the lines of force acting at the pelvic exit thus avoiding anatomical distortion, rather frequent in the techniques of repair in pelvic surgery. For these reasons the use of “tension free” meshes placed transperineally can be considered as a new technique in the correction of cystocele as well as Stress Urinary Incontinence (SUI).

The prosthesis according to the present invention has been positioned by the author using the technique described above in a large number of patients, about 400 with a follow-up of greater than 3 years, obtaining a cure rate of more than 90% with a low incidence of complications including: erosion (6%), dyspareunia (5%), pelvic pain (7%).

A further advantage of the prosthesis according to the present invention is an increase in the production of connective tissue between the pores of the surgically implanted mesh, creating a natural non-absorbable and elastic support which provides an effective support and prevents a further descent of the pelvic structures subject to prolapse and incontinence.

Moreover, the surgical application of the aforesaid prosthesis, which can be carried out both at an early and late stage of prolapse and incontinence, is able to correct all the existing defects, centrally and/or laterally and SUI and to prevent the potential appearance of further defects, so that a differential diagnosis of the position of the prolapse preparatory to surgical therapy is actually superfluous.

The present invention will now be described in a explanatory but not limitative document, according to the preferred modes of implementation, with particular reference to the diagrams attached, in which:

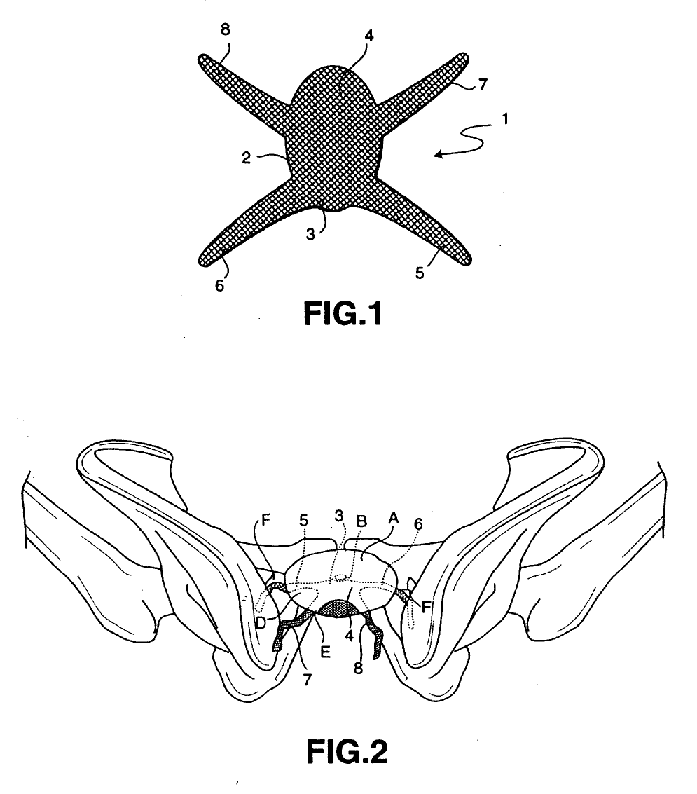

FIG. 1 shows a form of the prosthesis according to the present invention;

FIG. 2 shows a schematic design of the prosthesis of FIG. 1 applied to the female urogenital apparatus seen posteriorly;

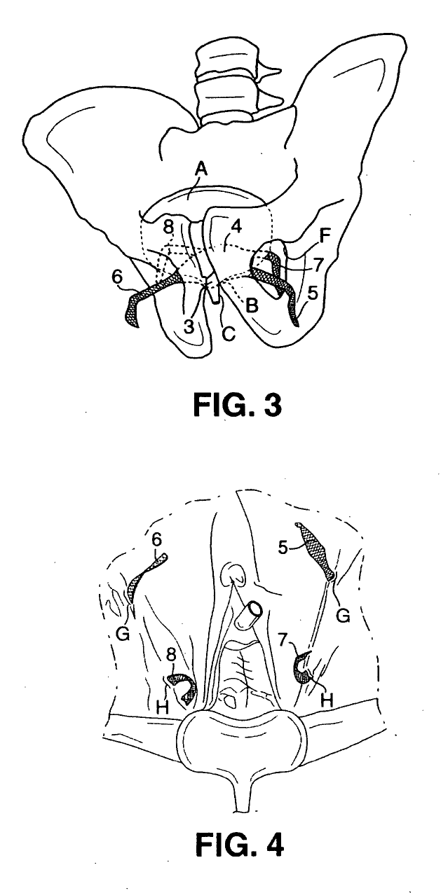

FIG. 3 shows a schematic design of the prosthesis of FIG. 1 applied to the female urogenital apparatus seen anteriorly;

FIG. 4 shows the final phase of the surgical technique for the application of the prosthesis of FIG. 1 in which the incisions for insertion of the arms of the prosthesis are illustrated.

Observing the figures of the attached diagrams, a form of realization of the prosthesis according to the invention is demonstrated, indicated generically with the reference number 1.

The prosthesis 1 constitutes a mesh made of non-absorbable or partially absorbable or biological material and includes a central body 2 with a first portion 3 designed to be placed under the bladder neck B and the middle urethra C, and a second portion 4 to be applied to the body D and the base E of the bladder beneath the inferior part of the bladder; the portions are nominated first and second as this is a preferred sequence of the direction of insertion of the prosthesis; a first pair of arms 5 and 6 which extend from each side of the first portion 3 of the central body 2, the said arms 5 and 6 designed to be placed in the obturator foramen F bilaterally and a second pair of arms 7 and 8 which extend from each side of the second section 4 of the central body 2, the said arms 7 and 8 designed to be applied to the perineum lateral to the labia major and penetrating the bulbocavernosus muscles (see FIG. 4 which demonstrates the incisions H through which pass arms 7 and 8). To facilitate the introduction and fixation of the 4 arms, the latter are covered with a nylon sheath which is then withdrawn once placed in position.

As described above, the surgical technique for the application of the prosthesis according with the present invention essentially consists of exposing the anterior vaginal wall and making a longitudinal incision from 1 cm below the external urethral meatus to the anterior vaginal fornix. The underlying pubocervical fascia is then detached, as far as the ascending branch of the ischiopubic and laterally to the lateral fornix. Two (2) lateral incisions (G) are then carried out 1 cm above the clitoris in the inguinal folds and a curved needle is passed through the obturator foramen (F) and exits at the sides of the bladder neck bilaterally. The apex of an arm of the prosthesis is then threaded into the needle and then pulled through the previous track, in this way positioning the first portion (3) of the central body (2) next to the bladder (A) under the bladder neck (B) and middle urethra (C) and the arms (5 and 6) in the obturator foramen (F) (see FIG. 4 which shows the incisions G through which arms 5 and 6 pass). Two (2) further incisions (H) of 1-2 cm are then executed laterally to the labia major at the level of the vaginal vestibule and with the same curved needle the track is penetrated, laterally to the bulbo-cavernosous muscle and the end of the needle is brought out 2 cm posterior to the lateral fornix, in order to position the second portion (4) of the central body (2) of the prosthesis (1) next to the bladder body (D) and the bladder base (E) and under the inferior part of the bladder and the arms (7 and 8) in the perineum laterally to the labia major and penetrating the perineum laterally to the bulbo-cavernosous muscle (see FIG. 4 which shows the incisions H through which arms 7 and 8 pass). A free flap of the mesh is placed under the inferior part of the bladder, and after having verified the lack of tension of the two anterior arms under the bladder neck using Metzenbaum scissors, the vaginal opening is closed with a continuous suture. With a combined maneuver, applying traction to the arms which have been placed outside and on the anterior vaginal wall (using two fingers placed in vagina), the mesh is positioned as gently as possible, the excess tissue is then excised and the 4 cutaneous openings are closed (G, H).

The present invention has been described in an explanatory but not limitative document, according to the preferred modes of realization, but it should be understood that variations and/or modifications may be added, without exceeding the relative protection, as defined by the attached claims.

1. Prosthesis to be used in the surgical treatment of urogenital prolapse and female urinary incontinence constituting a mesh made of non-absorbable or partially absorbable or biological material, the prosthesis comprising a central body having a first portion designed to be placed beneath the bladder under the bladder neck and middle urethra; a second portion is to be placed in relation to the bladder, Under the inferior part of the bladder, a first pair of arms which extend from each side of the first portion of the central body, the said arms designed to be placed in the obturator foramen bilaterally and a second pair of arms which extend from each side of the second portion of the central body, the said second arms designed to be placed on the perineum, laterally to the labia major and passing through the levator muscles.

2. Prosthesis according to claim 1 characterized in that the said arms of the said first and second pair are intended, at the respective ends not attached to the said central body, to be the means of attachment of curved surgical needles, preferably single use curved surgical needles.

3. Prosthesis according to claim 2 characterized in that the said means of attachment are non-reusable.

4. Prosthesis according to claim 2 characterized in that the said arms of the said first and second pair are covered with a nylon sheath.

5. Prosthesis according to claim 1 characterized in that the said arms of the first pair of arms are angled with respect to the central body of the prosthesis in the opposite direction to the preferred direction of insertion of this prosthesis.

6. Prosthesis according to claim 1 characterized in that the said arms of the second pair of arms are angled with respect to the central body of the prosthesis in the preferred direction of the insertion of this prosthesis.

7. Prosthesis according to claim 1 characterized in that the non-absorbable or partially absorbable synthetic mesh has bidirectional elastic distensibility and a resistance to traction greater than 10 KG/cm 2.

8. Prosthesis according to claim 7 characterized in that the mesh is between 0.5 mm and 0.9 mm thick.

9. Prosthesis according to claim 7 characterized in that the mesh is approximately 0.7 mm thick.

10. Prosthesis according claim 1 characterized in that the mesh is made of polypropylene.

11. Prosthesis according to claim 1 characterized in that the mesh is made of biological material centrally and synthetic material in the arms of both the first and second pairs of arms.

12. Prosthesis according to claim 2 characterized in that the said arms of the first pair of arms are angled with respect to the central body of the prosthesis in the opposite direction to the preferred direction of insertion of this prosthesis.

13. Prosthesis according to claim 3 characterized in that the said arms of the first pair of arms are angled with respect to the central body of the prosthesis in the opposite direction to the preferred direction of insertion of this prosthesis.

14. Prosthesis according to claim 4 characterized in that the said arms of the first pair of arms are angled with respect to the central body of the prosthesis in the opposite direction to the preferred direction of insertion of this prosthesis.
